(19) United States
(12) Patent Application Publication
(10) Pub. No.: US 2024/0408034 A1
(43) Pub. Date: Dec. 12, 2024

(54) METHOD FOR PREPARING LIPID NANOPARTICLES

(71) Applicant: leon-nanodrugs GmbH, Planegg (DE)

(72) Inventor: Frank STIENEKER, Planegg (DE)

(21) Appl. No.: 18/698,365

(22) PCT Filed: Oct. 6, 2022
(86) PCT No.: PCT/EP2022/077859

§ 371 Date: Apr. 4, 2024

(30) Foreign Application Priority Data
Oct. 6, 2021 (EP) 21201247.0

(51) Int. Cl. A61K 9/51; A61K 31/7105

(52) U.S. Cl. CPC A61K 9/5192; A61K 31/7105

(57) ABSTRACT

The invention provides methods of preparing lipid nanoparticles with biologically active agents associated with and/or encapsulated within the lipid nanoparticles. The method comprises the steps of (a) providing a first stream of a first liquid composition comprising an organic solution of one or more lipids; (b) providing a second stream of a second liquid composition comprising an aqueous solution of the biologically active agent; (c) mixing the first stream and the second stream such as to form a third stream of a third liquid composition comprising nascent lipid nanoparticles; (d) filling the third liquid composition into primary packaging containers without prior removal or addition of a constituent from or to the third liquid composition; and (e) subjecting the primary packaging containers to freeze drying such as to obtain a lyophilised composition. Steps (a) to (e) are conducted under aseptic conditions.

### TECHNICAL FIELD

The present invention relates to methods for preparing lipid nanoparticles for the delivery of biologically active agents to target tissues, and compositions comprising such lipid nanoparticles. The present disclosure also relates to methods which allow preparing lipid nanoparticles in an industrial GMP-compliant manner. Furthermore, the present disclosure relates to methods and compositions for storing lipid nanoparticles without substantial loss of the product quality and of the activity of the biologically active agent.

### Background of the Invention

Methods for preparing lipid nanoparticles comprising biologically active compounds, like e.g. nucleic acids, are known in the state of the art. Some methods basically comprise the mixture of a lipid solution with an aqueous solution of the nucleic acid. The methods often suffer from difficulties in scaling up of the process.

Ethanol dilution methods represent widely used methods for the production of lipid nanoparticles (see, e.g., Aldosari, B. N. et al., Lipid Nanoparticles as Delivery Systems for RNA-Based Vaccines. Pharmaceutics 2021, 13, 206; doi: 10.3390/pharmaceutics13020206). These methods are based on dissolving lipid compognents in ethanol, a water-miscible solvent, at specific concentrations. The alcoholic solution of lipids is then mixed with an aqueous solution. In principle, any suitable mixer may be used, including for example a T-shaped mixer.

WO2019077053A1 discloses a method for the production of RNA lipoplex particles based in the above principle. In a first step, a liposome dispersion is prepared by injecting a solution of liposome-forming lipids in ethanol into an aqueous phase, which results in the formation of liposomes. Subsequently the liposome dispersion is added to a solution comprising RNA under controlled mixing conditions. The mixing is supported by applying a flow rate, using syringes and Y-type or T-type mixing elements. After addition of a stabilizer or cryoprotectant, the RNA lipoplex composition is frozen, lyophilized or spray-dried for storage.

WO2018132909A1 discloses an example of a microfluidic mixing platform for the preparation of lipid nanoparticles. Such systems use small volume mixing technologies for generating the nanoparticulate formulations. Microfluidic platforms are made up of one or more inlets, microchannels, i.e., linear or curvilinear passages of about typically 80 to 1000 μm width, mixing regions, and one or more outlets. The mixing region is a downstream portion of the micromixer wherein the reagents are combined and the lipid nanoparticles are formed. The mixing platform can be used in any situation in which pressure is applied to push fluid through the fluid path to mix the contents, such as in the mechanical micromixer disclosed in WO2018006166A1. When using such microfluidic approaches, challenges in the upscaling process remain. In addition, the manufacture of the mixing platforms can cause difficulties.

Another approach for the production of lipid nanoparticles uses jet impingement reactors. The function of these reactors is based on the use of two fluid streams, at least one of which typically contains the active ingredient, that are injected into a reactor cavity and collide at a turbulent mixing zone, thereby creating the nanoparticles. In case one of the solvents contains a lipid, lipid nanoparticles can be produced with help of the jet impingement reactors which may, for example, be subsequently loaded with a biologically active compound, e.g., by pH shift.

PCT/EP2021/063855 and EP21172324 disclose methods for producing a liposome dispersion using a jet impingement reactor. A first fluid stream with organic solvent and second stream which is aqueous are ejected through pinholes into a reaction chamber and collide frontally. The first or the second stream comprises a lipid and the active pharmaceutical ingredient, which can be a small molecule, a nucleic acid or an antigen/polypeptide.

Nucleic acids can be used for the delivery of foreign genetic information into target cells and are often delivered in form of lipid nanoparticles. RNA, for example, may be delivered by so-called lipoplexes formulations. Lipoplexes are liposome-based formulations that form upon electrostatic interaction of cationic liposomes with RNAs. An overview of cationic lipids that can be used for the production of cationic liposomes can be found in Ponti 2021 (see e.g. Ponti F. et al., Cationic lipids for gene delivery: many players, one goal. Chemistry and Physics of Lipids 2021, doi: 10.1016/j.chemphyslip.2020.105032). However, it remains a challenge to obtain functional particles with precise and reproducible physico-chemical features and improved biological activity. Furthermore, liposome-based formulations are often characterized by their poor reproducibility and scalability. Therefore, the development of methods for GMP-compliant manufacturing of injectable RNA lipoplex particle formulations with a long shelf-life is an unmet need.

In addition, the stability of liposome-based formulations depends on the storage conditions and often requires deep-freeze temperatures and an uninterrupted cold chain during long-term storage and shipment to the destination of use.

It is thus an object of the present invention to provide stable lipid nanoparticle compositions that are easy to ship and store, and methods for their efficient production. Furthermore, the methods should be reproducable, easily scalable, easy to handle and overcome the other disadvantages of the prior art A specific object of the invention is to provide methods for preparing stable injectable compositions of lipid nanoparticles that are efficient and readily conducted under aseptic conditions. Another object of the present invention is to provide ready to use and administer compositions of lipid nanoparticles. Further objects of the invention will be clear on the basis of the following description of the invention, examples and claims.

### SUMMARY OF THE INVENTION

In a first aspect, the invention relates to a method of preparing a composition comprising lipid nanoparticles and a biologically active agent selected from oligo- and polynucleotides. The biologically active agent is associated with and/or encapsulated within the lipid nanoparticles. The method comprises the steps of (a) providing a first stream of a first liquid composition, wherein the first liquid composition comprises an organic solution of one or more lipids; (b) providing a second stream of a second liquid composition, wherein the second liquid composition comprises an aqueous solution of the biologically active agent; (c) mixing the first stream and the second stream such as to form a third stream of a third liquid composition, wherein the third liquid composition comprises nascent lipid nanoparticles comprising the one or more lipids; (d) directly filling the third liquid composition into a primary packaging container; and (e) subjecting the primary packaging container to freeze drying such as to obtain a lyophilised composition. Between step (c) and step (d) no step of removal or addition of a constituent from or to the third liquid composition is conducted. Moreover, steps (a) to (e) are conducted under aseptic conditions.

The method may comprise a further step of (f) reconstituting the lyophilised composition obtained in step (e) by combining it with a liquid reconstitution solvent such as to obtain a reconstituted liquid composition comprising the lipid nanoparticles and the biologically active agent associated with and/or encapsulated within the lipid nanoparticles. In this embodiment, the liquid reconstitution solvent is preferably a buffered aqueous liquid.

In a further aspect, the present invention provides a primary packaging container comprising the lyophilised composition as obtained in step (e) of the method according to the invention.

In a further aspect, the invention provides a reconstituted lyophilised composition as obtained in step (f) of the method according to the invention.

In a yet further aspect, the invention provides a kit comprising at least a first kit component and a second kit component, wherein the first kit component comprises a primary packaging container according to the invention, and wherein the second kit component comprises an amount of the liquid reconstitution solvent according to the method of the invention.

In yet a further aspect, the invention provides the use of the primary packaging container or of the reconstituted lyophilised composition according or of the kit according to the invention as a medicament.

### DESCRIPTION OF THE DRAWINGS

FIG. 1 shows a schematic overview according to one embodiment of the procedure for preparing a lipid nanoparticle composition.

FIG. 2 shows schematically the reconstitution step of one embodiment of the procedure for preparing a lipid nanoparticle composition.

FIG. 3, which is not to scale, depicts a jet impingement reactor used in one of the embodiments of the method of the invention.

### DETAILED DESCRIPTION OF THE INVENTION

### Definitions

Unless defined otherwise, or unless the specific context indicates or requires otherwise, all technical terms used herein have the same meaning as is commonly understood by a person skilled in the relevant technical field. ‘A’ or ‘an’ does not exclude a plurality; i.e. the singular forms ‘a’, ‘an’ and ‘the’ should be understood as to include plural referents unless the context clearly indicates or requires otherwise. The terms ‘a’, ‘an’ and ‘the’ hence have the same meaning as ‘at least one’ or as ‘one or more’ unless defined otherwise. For example, reference to ‘an ingredient’ includes mixtures of ingredients, and the like.

Unless the context clearly indicates or requires otherwise, the words ‘comprise’, ‘comprises’ and ‘comprising’ and similar expressions are to be construed in an open and inclusive sense, as ‘including, but not limited to’ in this description and in the claims.

The terms ‘substantially consist of’ essentially consist of mean that no further components are added to a composition or dosage form other than those listed. Nevertheless, very small amounts of other materials may potentially be present, such as material inherent impurities.

Further terms and expressions will be defined or explained in their context below.

In a first aspect, the invention provides a method of preparing a composition comprising lipid nanoparticles and a biologically active agent selected from oligo- and polynucleotides, wherein the biologically active agent is associated with and/or encapsulated within the lipid nanoparticles, comprising the steps of (a) providing a first stream of a first liquid composition, wherein the first liquid composition comprises an organic solution of one or more lipids; (b) providing a second stream of a second liquid composition, wherein the second liquid composition comprises an aqueous solution of the biologically active agent; (c) mixing the first stream and the second stream such as to form a third stream of a third liquid composition, wherein the third liquid composition comprises nascent lipid nanoparticles comprising the one or more lipids; (d) directly filling the third liquid composition into primary packaging containers; and (e) subjecting the primary packaging containers to freeze drying such as to obtain a lyophilised composition; wherein between step (c) and step (d) no step of removal or addition of a constituent from or to the third liquid composition is conducted; and wherein steps (a) to (e) are conducted under aseptic conditions.

The lipid nanoparticles produced by the method of the invention are nanoscale particles comprising at least one amphiphilic lipid. In other words, the produced nanoparticles are lipid-based nanoparticles. A skilled artisan would appreciate that a “nanoparticle”, also referred to as an ultrafine particle, is usually defined as a particle smaller than about 1000 nm. In some embodiments, the lipid nanoparticles produced by the method of the invention are selected from the group of unilamellar liposomes (liposomes having a single lipid bilayer), multilamellar liposomes, multivesicular liposomes, single bilayer liposomes, double bilayer liposomes, lipid complexes, lipoplexes, polyplexes (such as lipopolyplexes), solid lipid nanoparticles. In some embodiments, the lipid nanoparticles produced by the method of the invention are a combination of one or more types of lipid nanoparticles listed above.

A skilled artisan will appreciate that various types of lipid nanoparticles are currently used for clinical purposes or are in clinical development phases. Many of these may be produced by the methods disclosed herein.

The composition produced by the method of the invention further comprises a biologically active agent selected from oligo- and polynucleotides. The oligo- and polynucleotides are nucleotides made of deoxyribonucleic acid (DNA) or ribonucleic acid (RNA). The term “RNA”, also referred to as ribonucleic acid, relates to a nucleic acid molecule which includes ribonucleotide residues. The term “siRNA”, also referred to as short interfering RNA or silencing RNA, relates to a class of double-stranded non-coding RNA molecules, having a typical length of 20-27 base pairs. The term “mRNA” is also known as messenger RNA and relates to single-stranded molecules of RNA that correspond to the genetic sequence of a gene. The term “DNA”, also referred to as deoxyribonucleic acid, relates to a nucleic acid molecule which includes deoxyribonucleotide residues.

As used herein, oligonucleotides typically contain about 10 to about 100 nucleotides and polynucleotides typically contain more than about 100 nucleotides. In a specific embodiment, the oligo- and polynucleotides are plasmids or vectors, i.e., circular nucleotides, preferably double-stranded DNA plasmids.

In a preferred embodiment, the biologically active agent is an RNA molecule. In other words, the oligo- or polynucleotides are RNA molecules. In a more specific embodiment, the RNA molecule is selected from siRNA and mRNA. The mRNA is optionally a modified mRNA (modRNA), i.e., a synthetic mRNA in which some nucleosides are replaced by other naturally modified nucleosides or by synthetic nucleoside analogues. The use of modRNA may be advantageous since it decreases the immonugenicity of the RNA.

In an alternative embodiment, the composition produced by the method of the invention comprises a biologically active agent selected from peptides, polypeptides or proteins that preferably represent an antigens.

According to the invention, the biologically active agent is associated with and/or encapsulated within the lipid nanoparticles. This includes a broad variety of structures of a nanoparticle with an oligo- and polynucleotide that can be formed during the preparation of the composition. In some embodiments, the oligo- and polynucleotide exists within solvent-filled cavities of lipid nanoparticles; or the oligo- and polynucleotide is substantially lipid-associated. Mixtures of both forms can also be present. In some embodiments, multilamellar structures in which the oligo- and polynucleotide molecule, preferably the oligonucleotide molecule, is sandwiched between bilayer lipid assemblies are present.

In step (a) according to the method of the invention, a first stream of a first liquid composition is provided. The first liquid composition comprises an organic solution of one or more lipids. The one or more lipids preferably represent, or include, at least one amphiphilic lipid that is capable of forming a lipid bilayer in an aqeuous environment.

Any organic solvents known to the skilled artisan to be suitable for dissolving lipids for the preparation of lipid nanoparticles can be used in the methods disclosed herein. In some embodiments, the organic solvent is selected from the group of an alcohol, a ketone, a halogenated solvent, an amide, and an ether. In some embodiments, the organic solvent is selected from the group comprising ethanol, ethylene, bromide, butanol, acetone, chloroform, 2-ethylhexanol methylethylketone, ethylene chloride, isobutanol, methylisobutylketone, dichloromethane, isopropanol, methylisopropylketone, tetrachloroethylene methanol, mesityl oxide, carbon tetrachloride, propanol, trichloroethylene, propylene glycol, 1,4-dioxane butyl ether, ethyl ether, dimethylformamide, diisopropyl ether, dimethyl sulfoxide, tetrahydrofuran, tert-butyl methyl ether, hydrocarbons, aromatic hydrocarbons, cyclohexane, toluene, hexane, and xylene. In some embodiments, the organic solution comprises one organic solvent, preferably chosen of the above list. In some embodiments, the organic solution comprises a mixture of two or more organic solvents, preferably two different organic solvents, preferably chosen from the above list. In a particularly preferred embodiment, the organic solvent comprised in the first liquid composition is water-miscible and preferably selected from ethanol, methanol, acetone, acetonitrile, acetic acid, formic acid, n-butanol, trifluoroacetic acid, acetaldehyde, butanol, ethylamine and any combinations thereof. One of the preferred organic solvents is ethanol. Also preferred are embodiments in which organic solvents that are not water-miscible are absent or substantially absent in the first liquid composition.

In the context of the invention, an organic solution is a solution whose solvent or solvent mixture is at least predominantly organic. Possibly, small amounts of water may also be present. In one of the preferred embodiments, the organic solution is essentially free of water, such that any traces or residual amounts of water that may be present are technically substantially irrelevant.

As mentioned, any lipid known to the skilled artisan to be suitable for the preparation of lipid nanoparticles can be used in the methods disclosed herein. In some embodiments, the first liquid composition comprises an amphiphilic lipid capable of forming, or participating in the formation of a bilayer. For example, such lipid may be selected from the group of phosphatidylcholine, HSPC (hydrogenated soy phosphatidylcholine), DSPE (distearoyl-sn-glycero-phosphoethanolamine), DSPC (distearoylphosphatidylcholine), DOPC (dioleoylphosphatidylcholine), DPPG (dipalmitoylphosphatidylglycerol), EPC (egg phosphatidylcholine), DOPS (dioleoyl-phosphatidylserine), POPC (palmitoyloleoylphosphatidylcholine), SM (sphingomyelin), DMPC (dimyristoyl phosphatidylcholine), DOTAP (1,2-dioleoyloxy-3-[trimethylammonium]-propane), DMPG (dimyristoyl phosphatidylglycerol), DSPG (distearoylphosphatidylglycerol), DEPC (dierucoylphosphatidylcholine), DOPE (dioleoly-sn-glycero-phophoethanolamine), dipalmitoyl phosphatidylcholine (DPPC), methoxy polyethylene glycol (mPEG), mPEG-distearoyl phosphatidylethanolamine lipid conjugate, other phospholipids covalently bound to mPEG, cardiolipin, DSPE-N-[amino(polyethylene glycol)-2000], or any combination thereof.

In step (b) of the method according to the invention, a second stream of a second liquid composition is provided, wherein the second liquid composition comprises an aqueous solution of the biologically active agent. In other words, the second liquid composition comprises an aqueous solvent and the biologically active agent. As used herein, an aqueous solution whose liquid constituents predominantly consist of water. This does not entirely exclude that the composition comprises a small amount of an organic solvent. In one of the preferred embodiments, however, the aqueous solution is essentially free of organic solvents. In some alternative embodiments, the second liquid composition further comprises a water-miscible organic solvent. In a specific embodiment, the organic solvent is ethanol. In one embodiment, the aqueous solution has an acidic pH, i.e., below pH 7, or has a pH in the range of 4 to 6.5. In one embodiment, the aqueous phase comprises one or more pH-modifying agents, such as buffer components. Further ingredients may be present in the aqueous solution, as described below.

In step (c) of the method according to the invention, the first stream and the second stream are mixed such as to form a third stream of a third liquid composition, wherein the third liquid composition comprises nascent lipid nanoparticles comprising the one or more lipids. Accordingly, the combining and mixing of the first and the second liquid streams leads to the formation of nascent lipid nanoparticles.

As used herein, nascent lipid nanoparticles are lipid nanoparticles whose structure may optionally be the same as that of the final lipid nanoparticles that are produced by the method of the invention; or the structure may still differ to some extent from that of the final lipid nanoparticles, for example due to the presence of some organic solvent in the third liquid composition, such that the nascent lipid nanoparticles represent precursers of the lipid nanoparticles. In other words, precursers of the lipid nanoparticles may have a preliminary form, structure or composition. In precursers or nascent lipid nanoparticles, the association and/or encapsulation of the active agent within the lipid nanoparticles may not be fully finalized. The composition and morphology may still change between step (c) and the following freeze drying steps (e) and/or a subsequent reconstitution step. In some specific embodiments, the third liquid composition comprises the biologically active agent in free form, i.e. not associated with the lipid nanoparticles.

In a preferred embodiment, the mixing is based on a continuous flow principle, where the two streams are conducted into a mixing device and are processed as they travel through the mixing device that has an outlet for the resulting third stream. This allows continuous and simultaneous production of the third liquid composition.

In step (d) of the method of the invention, the third liquid composition as obtained in step (c) is directly filled into primary packaging containers. Such primary packaging containers are known to the skilled artisan. They can be any containers suitable for the filling and storage of the third liquid composition. Preferably, the containers are suitable for freeze drying. In some embodiments, the primary packaging containers are selected from vials, syringes, dual chamber syringes and bags. As used herein, bags are any flexible and/or collapsible containers that are suitable as primary packaging for sterile injectable products. An advantage of dual chamber syringes in the context of the invention is that they offer the possibility of accommodating not only the lyophilised composition obtained in step (e), but also the liquid reconstitution solvent of step (f), which makes reconstitution and administration easy and less error-prone. In some embodiments, the primary packaging containers are made of glass or plastic.

In some particularly preferred embodiments, the primary packaging containers are provided in sterile form. In some preferred embodiments, the primary packaging containers are sterile vials or syringes made of glass or plastic. In some preferred embodiments, the primary packaging containers are bags made of plastic.

According to the invention, no step of removal or addition of a constituent from or to the third liquid composition is conducted between step (c) and step (d). In other words, the third liquid composition comprising the nascent lipid nanoparticles is filled directly into the final primary packaging container. No solvent removal or buffer exchange, e.g. by tangential flow filtration or other suitable methods, is performed prior to step (d) or between step (c) and step (d), respectively. This is in contrast to prior art methods that rely on the preparation of the lipid nanoparticles followed by the removal of organic solvent(s) and optionally also the partial exchange of the aqueous solvent by tangential flow filtration or dialysis, which lead to aqueous liquid composition comprising the lipid nanoparticles which are in principle ready-to-use, but which are often frozen for storage and shipment due to stability issues. The present invention uses a more streamlined, efficient process to arrive at a more advantageous product design which is also stable but does not require storage and shipment at temperatures substantially below 0° C.

In step (e) of the method of the invention, the primary packaging containers are subjected to freeze drying such as to obtain a lyophilised composition. The freeze drying step, also referred to as lyophilisation, converts the third liquid composition into a solid composition, i.e. the lyophilised composition, by removing the liquid constituents from the third liquid composition. As generally known in the art, freeze drying involves the freezing of a liquid composition followed by the evaporation of the liquid constituent(s) of that composition under decreased pressure to allow the frozen medium in the substance to sublimate directly from the solid phase to the gas phase. The freezing can for example use dry ice, liquid nitrogen emersion or can be done in a freezing tunnel.

Lyophilisation typically provides for higher stability and longer storage times compared to liquid formulations, and often allows storage over several months or years at room temperature or under moderate refrigeration. Compared to frozen liquid formulations of lipid nanoparticles, dry compositions are easier to ship and store due to their lower weight. Typically, lyophilised compositions are relatively porous and may be easily and rapidly reconstituted.

The lyophilised composition that is obtained in step (e) may comprise the lipid nanoparticles with the biologically active agent being associated with and/or encapsulated within the lipid nanoparticles, or it may comprise any precursers of such nanoparticles. Without wishing to be bound by theory, it is believed that the form, structure, size and composition of lipid nanoparticles in a solid environment may differ from their form, structure, size and composition after reconstitution with an aqeuous solvent or carrier.

According to the invention, steps (a) to (e) are conducted under aseptic conditions. As used herein, aseptic conditions mean that the starting materials, the equipment surfaces in direct contact with these materials or with the product, and the product containers are provided in sterile form, and that any manipulations of materials occurs under conditions that exclude or prevent microbiological contamination.

In some embodiments, steps (a) to (e) are conducted under sterile conditions. A skilled person will appreciate that sterile conditions are conditions where the environment and/or the equipment is entirely free of germs. In some embodiments, the preparation of aseptic and/or sterile conditions is done with help of UV light, filtration, radiation, like e.g., gamma radiation, use of barriers, like e.g., sterile gloves, gowns, or drapes, use of masks, use of clean room facilities, use of sterile portable equipment, like e.g. sterilized primary packaging containers, or use of sterilized liquids.

Carrying out the method of the invention under aseptic conditions is advantageous since a sterilisation step of the resulting third liquid composition can be omitted. This saves time, reduces potential loss of lipid nanoparticles and increases the product quality of the resulting nanoparticles, e.g. with regard to their size and variance, e.g. variance in types of particles or size distribution (polydispersity index PDI).

In some embodiments, the method of the invention further comprises a step of (f) reconstituting the lyophilised composition obtained in step (e) by combining it with a liquid reconstitution solvent such as to obtain a reconstituted liquid composition comprising the lipid nanoparticles and the biologically active agent associated with and/or encapsulated within the lipid nanoparticles. The term “reconstitute” or “reconstitution” relates to converting a dry composition or a concentrate into a ready-to-use liquid composition by adding a solvent such as water or a buffered aqueous solution.

As used herein, the liquid reconstitution solvent is a solvent that is able to convert the lyophilised composition into a ready-to-use liquid composition, such as a lipid nanoparticle dispersion or a lipid nanoparticle solution.

As used herein, a lipid nanoparticle dispersion is a liquid composition comprising dispersed lipid nanoparticles. Depending on the actual size of the lipid nanoparticles, a liquid composition comprising lipid nanoparticles may not represent a true solution, as may be indicated, for example, by a cloudy or even opaque appearance. However, it is also observed that often lipid nanoparticle dispersions are referred to as lipid nanoparticle solution. As used herein, and unless the context dictates otherwise, the expressions “lipid nanoparticle dispersion” and “lipid nanoparticle solution” are used synonymously.

In some embodiments, the liquid reconstitution solvent is selected from water, preferably water for injection (WFI), buffered aqueous liquid, saline solutions, such as a 0.9 wt-% sodium chloride solution, or any other solvent that is able to dissolve or disperse the lyophilised composition. In preferred embodiments, the liquid reconstitution solvent is a buffered aqueous liquid. In specific embodiments, the buffered aqueous liquid has a pH from about pH 5 to about pH 9.

Preferably, the liquid reconstitution solvent, or buffered aqueous liquid, is provided in sterile form. Moreover, it is preferred that also step (f) is conducted under aseptic conditions, such as to prevent the microbiological contamination of the reconstituted liquid composition.

The liquid reconstitution solvent may comprise one or more further excipients, in particular excipients that are acceptable for injectable use, optionally selected from surfactants, antioxidants, stabilisers, co-solvents, and preservatives.

In some embodiments, the lyophilised composition and the liquid reconstitution solvent are combined by adding the liquid reconstitution solvent into the primary packaging containers comprising the lyophilised composition. The primary packaging containers are optionally gently shaken after adding the liquid reconstitution solvent, preferably until the lyophilised composition is fully dissolved or dispersed.

Optionally, the liquid reconstitution solvent is provided along with the lyophilised composition, for example if a dual chamber syringe is used as primary packaging container for holding the lyophilised composition in one chamber of the syringe and the liquid reconstitution solvent in the other.

The reconstituted liquid composition comprises the lipid nanoparticles and the biologically active agent associated with and/or encapsulated within the lipid nanoparticles. Optionally, it is during the reconstitution step that the final lipid nanoparticles are formed from precursor structures that may have been present in the lyophilised composition; unless the lipid nanoparticles having their final size, form, structure and composition were already formed at an earlier process step and subsequently preserved through all further steps.

Optionally, all of the biologically active agent is present in the reconstituted liquid composition. Alternatively, some of the active agent may exist in free form, i.e. not being encapsulated or associated with the lipid particles. In some preferred embodiment, the content of free active agent in the reconstituted liquid composition is not more than 10 wt-% relative to the entire amount of active ingredient in the composition, or not more than 5 wt-%, or not more than 1 wt-%, respectively. In one of the preferred embodiments, the reconstituted lyophilised composition does not contain any significant amounts of free biologically active agent.

As mentioned, in one of the preferred embodiments, the biologically active agent is an RNA molecule, such as an siRNA or mRNA molecule. The mRNA may optionally be a modified mRNA. Preferably, the mRNA molecule comprises a nucleic acid sequence capable of expressing a peptide, polypeptide or a protein that represents an antigen. As used herein, representing an antigen means that the peptide, polypeptide or a protein is able to provoke an immune response. This is advantageous if the lipid nanoparticles according to the present invention are used for the therapy and/or prophylaxis of diseases. In some embodiments, the disease is selected from an infectious disease, a cancerous disease, or an autoimmune disease.

In some embodiments, the antigen is a tumour antigen, a bacterial antigen, a viral antigen, a fungus antigen, or a protozoal antigen. In specific embodiments, the antigen is a viral antigen, preferably a coronavirus antigen.

In some embodiments, the one or more lipids comprise a cationic or cationisable lipid. This means that the first liquid composition comprises at least one cationic or at least one cationisable lipid, or a mixture of at least one cationic and at least one cationisable lipid. In one of the preferred embodiments, the first liquid composition comprises only one cationic or cationisable lipid. As used herein, and unless the context dictates otherwise, a cationic lipid is a lipid comprising a positive charge in an aqueous environment of any pH, such as a lipid having a quaternary nitrogen atom (i.e. an ammonium moiety); whereas a cationisable lipid is a lipid comprising a positive charge only in an aqueous environment of neutral or acidic pH, such as a lipid representing a primary, secondary or tertiary amine.

In a specific embodiment, the cationic or cationisable lipid consists of three different domains, wherein the domains are a cationic headgroup, covalently bound through a linker to a hydrophobic tail. In one embodiment, the cationic headgroup is selected from quaternary ammonium salts, amines (primary, secondary, and tertiary), guanidine, heterocyclic compounds, and a combination thereof. In one embodiment, the linker is selected from ethers, esters, carbamates, and amides. In one embodiment, the hydrophobic tails is selected from aliphatic chains or cyclic (steroid-based) domains.

In specific embodiments, the cationic or cationisable lipid is selected from the group of N1-[2-((1S)-1-[(3-aminopropyl)amino]-4-[di(3-amino-propyl)amino]butylcarboxamido)ethyl]-3,4-di[oleyloxy]-benzamide (MVL5), N4-cholesteryl-spermine HCl salt (GL67), N-(4-carboxybenzyl)-N,N-dimethyl-2,3-bis(oleoyloxy)propan-1-aminium (DOBAQ), 1,2-distearoyl-3-dimethylammonium-propane (18:0 DAP), 1,2-dipalmitoyl-3-dimethylammonium-propane (16:0 DAP), 1,2-dimyristoyl-3-dimethylammonium-propane (14:0 DAP), 1,2-dioleoyl-3-dimethylammonium-propane (DODAP) (18:1 DAP), 1,2-dioleyloxy-3-dimethylaminopropane (DODMA), 1,2-di-O-octadecenyl-3-trimethylammonium propane (chloride salt) (DOTMA), 1,2-dioleoyloxy-3-trimethylammonium-propane (chloride salt) (18:1 TAP or DOTAP), 1,2-stearoyl-3-trimethylammonium-propane (chloride salt) (18:0 TAP), 1,2-dipalmitoyl-3-trimethyl-ammonium-propane (chloride salt) (16:0 TAP), 1,2-dimyristoyl-3-trimethylammonium-propane (chloride salt) (14:0 TAP), dimethyldioctadecylammonium (bromide salt) (18:0 DDAB), 1,2-dimyristoleoyl-sn-glycero-3-ethylphosphocholine (Tf salt) (14:1 EPC Tf Salt), 1-palmitoyl-2-oleoyl-sn-glycero-3-ethylphosphocholine (chloride salt) (16:0-18:1 EPC Cl salt), 1,2-dioleoyl-sn-glycero-3-ethylphosphocholine (chloride salt) (18:1 EPC Cl salt), 1,2-distearoyl-sn-glycero-3-ethylphosphocholine (chloride salt) (18:0 EPC Cl salt), 1,2-dipalmitoyl-sn-glycero-3-ethylphosphocholine (chloride salt) (16:0 EPC Cl salt), 1,2-dimyristoyl-sn-glycero-3-ethylphosphocholine (chloride salt) (14:0 EPC Cl salt), 1,2-dilauroyl-sn-glycero-3-ethylphosphocholine (chloride salt) (12:0 EPC Cl salt), O,O′-ditetradecanoyl-N-(α-trimethylammonioacetyl)diethanolamine chloride (DC-6-14), 3ß-[N-(N′,N′-dimethylaminoethane)-carbamoyl]cholesterol hydrochloride (DC-cholesterol-HCl), 4-(dimethylamino)-butanoic acid, (10Z,13Z)-1-(9Z,12Z)-9,12-octadecadien-1-yl-10,13-nonadecadien-1-yl ester (D-Lin-MC3-DMA), ([(4-hydroxybutyl)azanediyl]di(hexane-6,1-diyl) bis(2-hexyldecanoate)) (ALC-0315), 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), cetyl-trimethyl ammonium bromide (CTAB), penta-amine N15-cholesteryloxycarbonyl-3,712-triazapentadecane-115-diamine (CTAP), bis-guanidinium-spermidine-Chol (BGSC), bis-guanidinium-tren-Chol (BGTC), N,N-distearyl-N-methyl-N-2[N′-(N2-guanidino-L-lysinyl)] aminoethyl ammonium chloride (DSGLA), O-(2R-1,2-di-O-(1Z,9Z-octadecadienyl)-glycerol)-3-N-(bis-2-aminoethyl)carbamate) (BTCA), 1,2-dilinoleyloxy-3-dimethylaminopropane (DLinDMA), N-[6-amino-1-oxo-1-(N-tetradecylamino)hexan-(2S)-2-yl]-N′-{2-[N,N-bis(2-aminoethyl)-amino]ethyl}-2,2-ditetradecylpropandiamide (DiTT4), cholesteryloxypropan-1-amine (COPA), and cholesteryl-2-aminoethylcarbamate (CAEC). Optionally, the first liquid composition comprises a mixture of two or more of the above listed cationic or cationisable lipids. Cationic or cationisable lipids are capable of interacting with the negatively charged oligo- and polynucleotides and support the association and/or encapsulation of the nucleotides in or with the lipid nanoparticle.

In some embodiments, the one or more lipids comprise a PEGylated lipid. As used herein, PEG means polyethylene glycol, and a PEGylated lipid is a lipid that is conjugated with a PEG moiety. In specific embodiments, the PEGylated lipid is selected from the group of 1,2-dimyristoyl-rac-glycero-3-methoxypolyethylene glycol-2000 (DMG-PEG 2000), distearoyl-rac-glycerol-PEG2K (DSG-PEG 2000), 2 [(polyethylene glycol)-2000]-N,N-ditetradecylacetamid (ALC-0159), N-(carbonyl-methoxypolyethylenglycol 2000)-1,2-dimyristoyl-sn-glycero-3-phosphoethanolamine sodium salt (MPEG-2000-DMPE Na), N-(carbonyl-methoxypolyethylenglycol 750)-1,2-distearoyl-sn-glycero-3-phosphoethanolamine sodium salt (MPEG-750-DSPE), 1,2-distearoyl-sn-glycero-3-phosphoethanolamine-N-[amino(polyethylene glycol)-2000] (ammonium salt) (DSPE-PEG(2000)), 1,2-dipalmitoyl-sn-glycero-3-phosphoethanolamine-N-[methoxy(polyethylene glycol)-2000] (ammonium salt) (16:0 PEG2000 PE), 1,2-dimyristoyl-sn-glycero-3-phosphoethanolamine-N-[methoxy(polyethylene glycol)-2000] (ammonium salt) (14:0 PEG2000 PE), 1,2-dioleoyl-sn-glycero-3-phosphoethanolamine-N-[methoxy(polyethylene glycol)-2000] (ammonium salt) (18:1 PEG2000 PE), 1,2-distearoyl-sn-glycero-3-phosphoethanolamine-N-[methoxy(polyethylene glycol)-2000] (ammonium salt) (18:0 PEG2000 PE), 1,2-distearoyl-sn-glycero-3-phosphoethanolamine-N-[methoxy(polyethylene glycol)-5000] (ammonium salt) (18:0 PEG5000 PE), N-(carbonyl-methoxypolyethylenglycol 2000)-1,2-dipalmitoyl-sn-glycero-3-phosphoethanolamine sodium salt (MPEG-2000-DPPE Na). Optionally, the first liquid composition comprises a mixture of two or more of the above listed PEGylated lipids. In one of the preferred embodiments, the first liquid composition comprises only one PEGylated lipid. Optionally, the PEGylated lipid is the same as the cationic or cationisable lipid. In an alterative embodiment, the first liquid composition comprises one PEGylated lipid which is not cationic or cationisable, and one cationic or cationisable which is not PEGylated.

In some embodiments, the one or more lipids comprise a non-PEGylated zwitterionic lipid. In specific embodiments, the non-PEGylated zwitterionic lipid is selected from the group of 1,2-dimyristoyl-sn-glycero-3-phosphocholine (DMPC), 1,2-dioleoyl-sn-glycero-3-phosphoethanolamine (18:1 (A9-cis) PE or DOPE), 1,2-Dipalmitoyl-sn-glycero-3-phosphate (sodium salt) (DPPA, Na), 1-palmitoyl-2-oleoyl-sn-glycero-3-phosphocholine (POPC), 1,2-dipalmitoyl-sn-glycero-3-phosphocholine (16:0 PC or DPPC), 1,2-distearoyl-sn-glycero-3-phosphocholine (DSPC), 1,2-dimyristoyl-sn-glycero-3-phosphatidylethanolamine (DMPE), 1-palmitoyl-2-oleoyl-sn-glycero-3-phosphatidylethanolamine (1,2-POPE), 1,2-dioleoyl-sn-glycerol-3-phosphatidylcholine (DOPC), and trans-2-aminoacyclohexanol (TACH). Optionally, the first liquid composition comprises a mixture of two or more of the above listed non-PEGylated zwitterionic lipids. In one of the preferred embodiments, it comprises only one non-PEGylated zwitterionic lipid.

In some further preferred embodiments, the one or more lipids comprise cholesterol. Alternatively, a cholesterol derivative may be used. The incorporation of cholesterol is believed to stabilise the lipid bilayers that may be formed by one or more of the other species of amphilic lipids as described above.

In a further embodiment, the first liquid composition comprises an organic solution of a cationic or cationisable lipid, a PEGylated lipid, a non-PEGylated zwitterionic lipid and cholesterol.

As mentioned, it is preferred that the first liquid composition, which comprises the organic solution of the one or more lipids, comprises a water-miscible solvent. The water-miscible solvent is preferably selected from ethanol, methanol, acetone, acetonitrile, acetic acid, formic acid, trifluoroacetic acid, acetaldehyde, butanol, ethylamine, and any combinations thereof. One of the particularly preferred water-miscible solvents is ethanol.

The total concentration of lipid, or lipids, in the first liquid composition may, for example, be in the range from about 20 mg/mL to about 100 mg/mL. In some further preferred embodiments, the total lipid concentration is in the range from about 50 mg/mL to about 100 mg/mL. Such relatively high concentration is also preferred if the third liquid composition is, in step (d), filled into a primary packaging container which already contains some aqueous liquid, such as an aqueous buffer solution, i.e. representing a fourth liquid composition as will be described in more detail further below.

In some preferred embodiments, the first liquid composition essentially consists of a solution of the one or more lipids and optionally one or more further lipophilic excipients in the water-miscible solvent. As used herein, further lipophilic excipients represent pharmaceutically acceptable additives that may be useful in the final product and that are more readily dissolvable in the organic solvent(s) of the first liquid composition than in the at least predominantly aqueous solution of the second liquid composition. Potentially useful lipophilic excipients or additives may include, for example, lipophilic antioxidants or other lipophilic stabilisers or surfactants.

In some related embodiments, the first liquid composition essentially consists of a solution of a cationic or cationisable lipid, a PEGylated lipid, a non-PEGylated zwitterionic lipid, cholesterol, and optionally one or more further lipophilic excipients in ethanol. In this context, the cationic or cationisable lipid, the PEGylated lipid, and/or the non-PEGylated zwitterionic lipid may be selected from the respective compounds disclosed as optional or preferred examples from each of these types of lipids herein-above.

Also preferred are embodiments in which the first liquid composition essentially consists of a solution of the one or more lipids in a water-miscible solvent selected from ethanol, methanol, acetone, acetonitrile, acetic acid, formic acid, trifluoroacetic acid, acetaldehyde, butanol, ethylamine, and any combinations thereof. In a specific embodiment, the first liquid composition essentially consists of a solution of the one or more lipids in ethanol.

In some embodiments, the first liquid composition comprises:
from 40 to 60 mol % of a cationic or cationisable lipid;
from 0.5 to 2 mol % of a PEGylated lipid;
from 5 to 20 mol % of a non-PEGylated zwitterionic lipid; and
from 30 to 50 mol % of cholesterol;
   wherein the percentages are based on the total content of lipids in the first liquid composition.

In one of the preferred embodiments, the method of the invention is carried out at elevated pressure. As used herein, the term “elevated pressure” can be used interchangeably with high pressure, overpressure, or elevated hydrodynamic pressure, and refers to any pressure above the atmospheric pressure. In other word, the pressure of the first and second stream is measured in comparison to the atmospheric pressure and is expressed as gauge pressure, that is typically obtained from a pressure gauge that is in fluid connection with the respective fluid to be measured. Therefore, a pressure of, e.g., 0.1 bar is to be understood as 0.1 bar above the atmospheric pressure. In a further preferred embodiment, the first stream is provided in step (a) and/or the second stream is provided in step (b) at a pressure in the range of about 10 kPa (0.1 bar) to about 12,000 kPa (120 bar), more preferably in the range of about 100 kPa (1 bar) to about 4,000 kPa (40 bar). Another preferred pressure range is from about 50 kPa (0.5 bar) to about 2,000 kPa (20 bar).

In a further preferred embodiment of the method of the invention the first stream is provided in step (a) and/or the second stream is provided in step (b) at a flow rate in the range of about 1 to about 1,000 ml/min. Other preferred ranges of the flow rate are from about 5 to about 500 ml/min and from about 10 to about 300 ml/min, respectively. Preferably, the flow rate of the second stream is higher than the flow rate of the first stream. For example, the flow rate ratio between second stream and first stream may be higher than 1.2, or between 1.2 and 5, or between 1.2 and 3, respectively. According to some further preferred embodiments, the flow rate of the second stream in step (b) is from about 2 times to about 4 times higher than the flow rate of first stream in step (a). For example, the flow rate of the second stream in step (b) may be about 2, 2.5, 3, 3.5 or 4 times higher than the flow rate of first stream in step (a).

Like the preferences regarding the pressures, the preferred flow rates should also be understood as generally applicable and thus combinable with one another. In other words, there is also an option or even preference for an embodiment of the method in which the first and the second fluid are provides at a pressure in the range of about 0.1 to about 120 bar, in particular of about 1 to about 40 bar, and at a flow rate in the range of about 1 to about 1,000 ml/min, or about 5 to about 500 ml/min, or about 10 to 300 ml/min.

In some embodiments of the method of the invention, the second liquid composition comprises one or more pH-regulating agents. Suitable pH-regulating agents are well known to the skilled person and can be selected of an acid, a base, a salt, or a combination thereof. In further embodiments, the second liquid composition has a pH in the range of about 4 to about 6.5, such as from about 5 to about 6.5. In one embodiment, the one or more pH-regulating agent is a citrate buffer.

In some further embodiments, the second liquid composition comprises an osmotic agent. The osmotic agent may be an inorganic compound, such as a physiologically tolerable salt, for example sodium chloride or potassium chloride; or an organic compound such as a sugar or sugar alcohol. Optionally, the osmotic agent is selected from sodium chloride, potassium chloride, sorbitol, mannitol and sucrose.

In some further embodiments, the second liquid composition comprises a lyophilisation aid. In the context of the present invention, a lyophilisation aid is a pharmaceutical excipient that provides bulk to a lyophilised composition and often also increases the stability of the biologically active ingredient during freeze drying. Other expressions commonly used for lyophilisation aids include bulking agents, cryoprotectants and lyoprotectants. A lyophilisation may also substantially contribute to the osmotic pressure of a composition. Therefore, at least some lyophilisation aids would also qualify as osmotic agents.

Preferably, the lyophilisation aid is a carbohydrate selected from a monosaccharide, a disaccharide, a trisaccharide, a sugar alcohol, an oligosaccharide or its corresponding sugar alcohol, and a straight chain polyalcohol. More preferably, the lyophilisation aid is selected from sucrose, trehalose, mannitol, sorbitol, glucose, fructose, proline, sorbitol, glycine betaine, polyethylene glycol, starch and dextran. In one of the particularly preferred embodiments, the lyophilisation aid is selected from sucrose, trehalose, and mannitol.

As discussed in the context of aseptic conditions, it is highly preferred that the first liquid composition and/or the second liquid composition are sterile, i.e. they are provided in steps (a) and (b) in sterile form. It is also highly preferred that the primary packaging containers provided for step (d) are sterile.

According to some preferred embodiments, the third liquid composition obtained by mixing the first and the second liquid composition is suitable as such for directly performing the freeze drying step after the third composition has been filled into the primary packaging containers. Accordingly, the sterile primary packaging container may be empty when provided for being filled according to step (d).

In some alternative preferred embodiments, it may be useful to modify the third liquid composition before freeze drying. For this purpose, the primary packaging container may contain an amount of a fourth liquid composition when provided for filling according to step (d). Consequently, step (d) results in the mixing of the third and the fourth liquid composition such as to form a fifth liquid composition.

A function of the fourth liquid composition may be to dilute the third liquid composition, for example in order to make it more suitable for the subsequent lyophilisation step. It may, for example, be useful to reduce the concentration of organic solvent(s) in the product before it is lyophilised. Alternatively, or in addition, the fourth liquid composition may be useful for adding one or more further excipients to the third liquid composition. Moreover, the fourth liquid composition may be useful for changing the pH of the third liquid composition.

Accordingly, the fourth liquid composition may, for example, be an aqueous solution comprising one or more excipients selected from pH-regulating agents, osmotic agents, lyophilisation aids, or any combinations thereof.

Examples of potentially suitable pH-regulating agents, osmotic agents and lyophilisation aids have already been described as optional constituents of the second liquid composition. The same options and preferences provided in that context are also applicable with respect to any pH-regulating agents, osmotic agents and/or lyophilisation aids comprised in the fourth liquid composition.

If used, the constituents but also the amount of the fourth liquid composition that is provided in the primary packaging container for performing the filling step (d) may be selected to optimise the composition of the final (fifth) liquid composition with respect to its processability in a lyophilisation, or freeze drying, process; and/or these parameters may be selected to optimise the properties of a reconstituted liquid composition resulting from step (f). In some of the preferred embodiments, the amount of the fourth liquid composition present in the primary packaging container when provided for filling according to step (d) is from about 50 to about 150 wt. % relative to the amount of the third liquid composition filled into the primary packaging container in step (d). In other preferred embodiments, the amount of the fourth liquid composition is from about 75 to about 125 wt % relative to the amount of the third liquid composition.

According to some further embodiments, the resulting fifth liquid composition is an aqueous composition comprising lipid nanoparticles, further comprising from about 5 to about 20 vol. % organic solvent. In this context, and according to one of the preferences, the organic solvent is ethanol. In some related embodiments, other organic solvents are essentially absent from the fifth liquid composition.

Accordingly, the fourth liquid composition may be composed and measured such that its combination with the third liquid composition during the filling step (d) results in a fifth liquid composition having an ethanol content of about 5 to about 20 vol. %, or from about 5 to about 15 vol. %, respectively.

In some other preferred embodiments, the fifth liquid composition is an aqueous composition comprising from about 5 to about 35 wt %, such as from about 10 to about 25 wt % of a lyophilisation aid. As described above, such amount of lyophilisation aid in the fifth composition may result from the presence of such lyophilisation aid in the second liquid composition and/or in the fourth liquid composition.

As mentioned, one of the advantages of the method of the invention is that it more efficiently leads to a lyophilisable product containing lipid nanoparticles in that an intermediate step of tangential flow filtration or the like is avoided. The incorporation of at least some of the lyophilisation aid already in the second liquid composition, i.e. before the lipid nanoparticles are formed by mixing the first and the second liquid composition, appears to be useful towards this purpose.

These options are further illustrated by the following embodiments. In some embodiments, the second liquid composition essentially consists of an aqueous solution of the biologically active agent. In some further embodiments, the second liquid composition essentially consists of an aqueous solution of the biologically active agent and one or more pH-regulating agents, and optionally an osmotic agent. In these embodiments, the second liquid composition does not comprise a lyophilisation aid. In some further embodiments, the second liquid composition essentially consists of an aqueous solution of the biologically active agent and one or more pH-regulating agents, and optionally an osmotic agent and/or a lyophilisation aid.

In some embodiments, the primary packaging containers, preferably the sterile vials or syringes, are empty when provided for filling according to step (d). Thus, the third liquid composition is not diluted and no further constituents are added to it prior to step (e).

In a specific embodiment of the method of the invention, the second liquid composition essentially consists of an aqueous solution of the biologically active agent and the primary packaging containers contain an amount of a fourth liquid composition when provided for their filling according to step (d).

As mentioned, the mixing of the first and the second liquid compositions according to step (c) may be performed by any suitable mixing device. Preferably, the mixing device is a static mixing device. In this context, a static mixing device is characterised in that it has no moving parts or elements whose movement is required for mixing. Specific variants of static mixing devices include microfluidic mixing platforms, jet impingement reactors, T-piece mixers, Y-shaped mixers, or multi-inlet vortex mixers.

Accordingly, in some embodiments, the mixing of the first stream and the second stream in step c) is performed using mixing devices being selected from static mixers, microfluidic mixing platforms, jet impingement reactors, T-piece mixers, Y-shaped mixers, or multi-inlet vortex mixers. In a preferred embodiment, the mixing of the first stream and the second stream in step (c) is performed using a T-piece, a Y-piece, a static mixer, or a microfluidic mixer.

In some embodiments, the static mixer comprises a mixing chamber such that said streams collide with one another frontally in the mixing chamber into which each of the first and the second stream are injected through a nozzle, such that said streams collide with one another frontally in the mixing chamber.

In a further preferred embodiment, a jet impingement reactor, which is a special type of static mixing device, is used for carrying out the method of the invention. The function of jet impingement reactors involves the injection of two fluid streams, e.g. the first stream and the second stream of the method of the invention, through nozzles into a reactor cavity such that the streams collide in a turbulent mixing zone. Preferably, the first and the second stream are injected from directly opposite positions of the reactor, preferably such that the streams collide substantially frontally, i.e. in an angle of substantially about 180°.

In a preferred embodiment, the mixing chamber is part of a jet impingement reactor, defined by an interior surface of a mixing chamber wall, wherein the mixing chamber has a substantially spheroidal overall shape. More preferably, the mixing chamber comprises (a) a first and a second fluid inlet, wherein the first and the second fluid inlet are arranged at opposite positions on a first central axis of the reaction chamber such as to point at one another, and wherein each of the first and the second fluid inlet comprises a nozzle; and (b) a fluid outlet arranged at a third position, said third position being located on a second central axis of said chamber, the second central axis being perpendicular to the first central axis. In this preferred embodiment, the distance between the nozzle of the first fluid inlet and the nozzle of the second fluid inlet is the same or smaller than the diameter of the mixing chamber along the first central axis. In specific related embodiments, the nozzle is a plain orifice nozzle.

The use of a jet impingement reactor for the mixing step (c) and/or the use of a reactor with a mixing chamber having a substantially spheroidal overall shape as described above provides a more precise control over the mixing and nanoparticle formation process. The properties (size, variants, PDI) of the lidpid nanoparticles can be defined more narrowly. Furthermore, a better scalability of the production process is provided, which is of advantage when the process scaled up to larger batch sizes of the product or when the lipid nanoparticles are prepared in an industrial GMP-compliant manner. The inventors have found that the use of such reactor allows the processing of relatively concentrated liquids, i.e. wherein the first liquid composition is an ethanolic lipid solution having a relatively high lipid concentration as described above, and/or wherein the second liquid composition already comprises some or all of the lyophilisation aid that may be desired for step (e), such that the third liquid composition can be lyophilised as such or after only moderate dilution by a fourth liquid composition, in any case without an interim tangential flow filtration step.

The solvent removal and rebuffering may also partially be achieved by the freeze drying step (e) itself, of the method of the invention. Thus, the lipid nanoparticles can be stored without substantial loss of the product quality and of the activity of the biologically active agent. Furthermore, the method can be conducted as a continuous process up to the filling of the primary packaging containers.

In a further aspect, the invention provides a primary packaging container comprising the lyophilised composition as obtained in step (e) of the method according to the invention. Using this product design which incorporates the lipid nanoparticles in lyophilised form brings about a higher stability and longer storage time, e.g. several months or even years compared to liquid formulations of lipid nanoparticles, e.g. frozen vaccine formulations. Such products are easier to ship and store due to their low weight Additionally, the lipid nanoparticles can be provided in a concentrated form after reconstitution.

In a further aspect, the invention provides a reconstituted lyophilised composition as obtained in step (f) of the method according to the invention. The reconstituted lyophilised composition comprises the lipid nanoparticles and the biologically active agent associated with and/or encapsulated within the lipid nanoparticles. The lipid nanoparticles have their final form, structure, size and composition. In some embodiments, the reconstituted liquid composition comprises not more than 10 wt-% free biologically active agent, or not more than 5 wt.-%, or not more than 1 wt-%, relative to the total amount of active agent in the composition.

In a further aspect, the invention provides a kit comprising at least a first kit component and a second kit component. The first kit component comprises a primary packaging container according to the invention. The second kit component comprises an amount of the liquid reconstitution solvent as described above. The provision of the kit is advantageous in that it facilitates the preparation of the ready-to-use reconstituted liquid composition directly at the point of care, i.e. at the place of use, and helps to avoid reconstitution errors. After reconstitution with the second kit component, the ready-to-use composition comprising the lipid nanoparticles with the biologically active agent that is associated with and/or encapsulated within the lipid nanoparticles may be directly administered to subjects such as patients.

In an even further aspect, the invention provides a use of the primary packaging container comprising the lyophilised composition as obtained in step (e) or of the reconstituted lyophilised composition as obtained in step (f) or of the kit according to the invention as a medicament. Expressed in other words, the primary packaging container filled with the lyophilised composition, the reconstituted lyophilised composition or the kit as described herein may be used in the manufacture of a medicament. The medicament may, in some preferred embodiments, represent a vaccine. For example, the vaccine may be a COVID vaccine.

Accordingly, the medicament may be for the prevention of an infection, such as a COVID or COVID-19 infection.

The following list of numbered items are further embodiments comprised in the present disclosure:
1. A method of preparing a composition comprising lipid nanoparticles and a biologically active agent selected from oligo- and polynucleotides, wherein the biologically active agent is associated with and/or encapsulated within the lipid nanoparticles, comprising the steps of:
   (a) providing a first stream of a first liquid composition, wherein the first liquid composition comprises an organic solution of one or more lipids;
   (b) providing a second stream of a second liquid composition, wherein the second liquid composition comprises an aqueous solution of the biologically active agent; (c) mixing the first stream and the second stream such as to form a third stream of a third liquid composition, wherein the third liquid composition comprises nascent lipid nanoparticles comprising the one or more lipids;
   (d) filling the third liquid composition into primary packaging containers; and
   (e) subjecting the primary packaging containers to freeze drying such as to obtain a lyophilised composition;
   wherein step (d) is conducted without prior removal or addition of a constituent from or to the third liquid composition; and
   wherein steps (a) to (e) are conducted under aseptic conditions.
2. The method of item 1, further comprising a step of
   (f) reconstituting the lyophilised composition obtained in step (e) by combining it with a liquid reconstitution solvent such as to obtain a reconstituted liquid composition comprising the lipid nanoparticles and the biologically active agent associated with and/or encapsulated within the lipid nanoparticles, wherein the liquid reconstitution solvent is preferably a buffered aqueous liquid.
3. The method of any one of the preceding items, wherein the biologically active agent is an RNA molecule, wherein the RNA molecule is preferably selected from siRNA and mRNA, and wherein the mRNA is optionally a modified RNA.
4. The method of any one of the preceding items, wherein the one or more lipids comprise:
   a cationic or cationisable lipid, wherein the cationic or cationisable lipid is preferably selected from the group of N1-[2-((1S)-1-[(3-aminopropyl)amino]-4-[di(3-amino-propyl)amino]butylcarboxamido)ethyl]-3,4-di[oleyloxy]-benzamide (MVL5), N4-cholesteryl-spermine HCl salt (GL67), N-(4-carboxybenzyl)-N,N-dimethyl-2,3-bis(oleoyloxy)propan-1-aminium (DOBAQ), 1,2-distearoyl-3-dimethylammonium-propane (18:0 DAP), 1,2-dipalmitoyl-3-dimethylammonium-propane (16:0 DAP), 1,2-dimyristoyl-3-dimethylammonium-propane (14:0 DAP), 1,2-dioleoyl-3-dimethylammonium-propane (DODAP) (18:1 DAP), 1,2-dioleyloxy-3-dimethylaminopropane (DODMA), 1,2-di-O-octadecenyl-3-trimethylammonium propane (chloride salt) (DOTMA), 1,2-dioleoyloxy-3-trimethylammonium-propane (chloride salt) (18:1 TAP or DOTAP), 1,2-stearoyl-3-trimethylammonium-propane (chloride salt) (18:0 TAP), 1,2-dipalmitoyl-3-trimethylammonium-propane (chloride salt) (16:0 TAP), 1,2-dimyristoyl-3-trimethylammonium-propane (chloride salt) (14:0 TAP), dimethyldioctadecylammonium (bromide salt) (18:0 DDAB), 1,2-dimyristoleoyl-sn-glycero-3-ethylphosphocholine (Tf salt) (14:1 EPC Tf Salt), 1-palmitoyl-2-oleoyl-sn-glycero-3-ethylphosphocholine (chloride salt) (16:0-18:1 EPC Cl salt), 1,2-dioleoyl-sn-glycero-3-ethylphosphocholine (chloride salt) (18:1 EPC Cl salt), 1,2-distearoyl-sn-glycero-3-ethylphosphocholine (chloride salt) (18:0 EPC Cl salt), 1,2-dipalmitoyl-sn-glycero-3-ethylphosphocholine (chloride salt) (16:0 EPC Cl salt), 1,2-dimyristoyl-sn-glycero-3-ethylphosphocholine (chloride salt) (14:0 EPC Cl salt), 1,2-dilauroyl-sn-glycero-3-ethylphosphocholine (chloride salt) (12:0 EPC Cl Salt), O,O′-ditetradecanoyl-N-(α-trimethylammonioacetyl)diethanolamine chloride (DC-6-14), 3ß-[N-(N′,N′-dimethylaminoethane)-carbamoyl]cholesterol hydrochloride (DC-cholesterol-HCl), 4-(dimethylamino)-butanoic acid, (10Z,13Z)-1-(9Z,12Z)-9,12-octadecadien-1-yl-10,13-nonadecadien-1-yl ester (D-Lin-MC3-DMA), ([(4-hydroxybutyl)azanediyl]di(hexane-6,1-diyl) bis(2-hexyldecanoate)) (ALC-0315), 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), cetyl-trimethyl ammonium bromide (CTAB), penta-amine N15-cholesteryloxycarbonyl-3,712-triazapentadecane-115-diamine (CTAP), bis-guanidinium-spermidine-Chol (BGSC), bis-guanidinium-tren-Chol (BGTC), N,N-distearyl-N-methyl-N-2[N′-(N2-guanidino-L-lysinyl)] aminoethyl ammonium chloride (DSGLA), O-(2R-1,2-di-O-(1Z,9Z-octadecadienyl)-glycerol)-3-N-(bis-2-aminoethyl)carbamate) (BTCA), 1,2-dilinoleyloxy-3-dimethylaminopropane (DLinDMA), N-[6-amino-1-oxo-1-(N-tetradecylamino)hexan-(2S)-2-yl]-N′-{2-[N,N-bis(2-aminoethyl)-amino]ethyl}-2,2-ditetradecylpropandiamide (DiTT4), cholesteryloxypropan-1-amine (COPA), and cholesteryl-2-aminoethylcarbamate (CAEC);
   a PEGylated lipid, wherein the PEGylated lipid is preferably selected from the group of 1,2-dimyristoyl-rac-glycero-3-methoxypolyethylene glycol-2000 (DMG-PEG 2000), distearoyl-rac-glycerol-PEG2K (DSG-PEG 2000), 2 [(polyethylene glycol)-2000]-N,N-ditetradecylacetamid (ALC-0159), N-(carbonyl-methoxypolyethylenglycol 2000)-1,2-dimyristoyl-sn-glycero-3-phosphoethanolamine sodium salt (MPEG-2000-DMPE Na), N-(carbonyl-methoxypolyethylenglycol 750)-1,2-distearoyl-sn-glycero-3-phosphoethanolamine sodium salt (MPEG-750-DSPE), 1,2-distearoyl-sn-glycero-3-phosphoethanolamine-N-[amino(polyethylene glycol)-2000] (ammonium salt) (DSPE-PEG(2000)), 1,2-dipalmitoyl-sn-glycero-3-phosphoethanolamine-N-[methoxy(polyethylene glycol)-2000] (ammonium salt) (16:0 PEG2000 PE), 1,2-dimyristoyl-sn-glycero-3-phosphoethanolamine-N-[methoxy(polyethylene glycol)-2000](ammonium salt) (14:0 PEG2000 PE), 1,2-dioleoyl-sn-glycero-3-phosphoethanolamine-N-[methoxy(polyethylene glycol)-2000] (ammonium salt) (18:1 PEG2000 PE), 1,2-distearoyl-sn-glycero-3-phosphoethanolamine-N-[methoxy(polyethylene glycol)-2000](ammonium salt) (18:0 PEG2000 PE), 1,2-distearoyl-sn-glycero-3-phosphoethanolamine-N-[methoxy(polyethylene glycol)-5000] (ammonium salt) (18:0 PEG5000 PE), N-(carbonyl-methoxypolyethylenglycol 2000)-1,2-dipalmitoyl-sn-glycero-3-phosphoethanolamine sodium salt (MPEG-2000-DPPE Na);
   a non-PEGylated zwitterionic lipid, wherein the non-PEGylated zwitterionic lipid is preferably selected from the group of 1,2-dimyristoyl-sn-glycero-3-phosphocholine (DMPC), 1,2-dioleoyl-sn-glycero-3-phosphoethanolamine (18:1 (A9-cis) PE or DOPE), 1,2-Dipalmitoyl-sn-glycero-3-phosphate (sodium salt) (DPPA, Na), 1-palmitoyl-2-oleoyl-sn-glycero-3-phosphocholine (POPC), 1,2-dipalmitoyl-sn-glycero-3-phosphocholine (16:0 PC or DPPC), 1,2-distearoyl-sn-glycero-3-phosphocholine (DSPC), 1,2-dimyristoyl-sn-glycero-3-phosphatidylethanolamine (DMPE), 1-palmitoyl-2-oleoyl-sn-glycero-3-phosphatidylethanolamine (1,2-POPE), cholesterol 3-sulfate (sodium salt) (SCS), 1,2-dioleoyl-sn-glycerol-3-phosphatidylcholine (DOPC), and trans-2-aminoacyclohexanol (TACH); and/or
   cholesterol.
5. The method of any one of the preceding items, wherein the first liquid composition comprises a water-miscible solvent, wherein the water-miscible solvent is preferably selected from ethanol, methanol, acetone, acetonitrile, acetic acid, formic acid, trifluoroacetic acid, acetaldehyde, n-butanol, ethylamine, and any combinations thereof.
6. The method of item 5, wherein the first liquid composition essentially consists of a solution of the one or more lipids and optionally one or more further lipophilic excipients in the water-miscible solvent
7. The method of any one of the preceding items, wherein the first stream is provided in step (a) and/or the second stream is provided in step (b) at a pressure in the range of 10 kPa (0.1 bar) to 12,000 kPa (120 bar) and/or wherein the first stream is provided in step (a) and/or the second stream is provided in step (b) at a flow rate in the range of 1 to 1,000 ml/min, and wherein the flow rate of the second stream is preferably higher than the flow rate of the first stream.
8. The method of any one of the preceding items, wherein the second liquid composition comprises an osmotic agent, and wherein the osmotic agent is optionally selected from sodium chloride, potassium chloride, sorbitol, mannitol, and sucrose and/or wherein the second liquid composition comprises a lyophilisation aid, and wherein the lyophilisation aid is optionally selected from sucrose, trehalose, mannitol, sorbitol, glucose, fructose, proline, glycine betaine, polyethylene glycol, starch and dextran.
9. The method of any one of the preceding items, wherein the primary packaging containers are empty when provided for filling according to step (d), or contain an amount of a fourth liquid composition when provided for their filling according to step (d), wherein the fourth liquid composition preferably is an aqueous solution comprising
   a pH-regulating agent;
   an osmotic agent; and/or
   a lyophilisation aid.
10. The method of any one of the preceding items, wherein the mixing of the first stream and the second stream in step (c) is performed using a T-piece, a Y-piece, a static mixer, or a microfluidic mixer.
11. The method of any one of the preceding items, wherein step (c) comprises the substeps of:
   (i) providing a mixing chamber (6), and
   (ii) injecting each of the first and the second stream through a nozzle (5) into the mixing chamber such that said streams collide with one another frontally.
12. The method of item 11, wherein the mixing chamber is part of a jet impingement reactor (1), defined by an interior surface (2) of a mixing chamber wall (3), the mixing chamber (6) having a substantially spheroidal overall shape and wherein the mixing chamber (6) preferably comprises
   (a) a first and a second fluid inlet (4), wherein the first and the second fluid inlet (4) are arranged at opposite positions on a first central axis (x) of the reaction chamber (6) such as to point at one another, and wherein each of the first and the second fluid inlet (4) comprises a nozzle (5); and
   (b) a fluid outlet (7) arranged at a third position, said third position being located on a second central axis (y) of said chamber (6), the second central axis (y) being perpendicular to the first central axis (x); and
   wherein the distance (d) between the nozzle (5) of the first fluid inlet (4) and the nozzle (5) of the second fluid inlet (4) is the same or smaller than the diameter of the mixing chamber (6) along the first central axis (x).
13. A primary packaging container comprising the lyophilised composition as obtained in step (e) of the method according to any of the items 1 to 12.
14. A reconstituted lyophilised composition as obtained in step (f) of the method according to any of the items 2 to 12.
15. A kit comprising at least a first kit component and a second kit component, wherein the first kit component comprises a primary packaging container according to item 13, and wherein the second kit component comprises an amount of the liquid reconstitution solvent according to any of the item 2 to 12.
16. Use of the primary packaging container according to item 13 or of the reconstituted lyophilised composition of item 14 or of the kit of item 15 as a medicament, wherein the medicament is preferably a vaccine.

The following examples serve to illustrate the invention, however should not to be understood as restricting the scope of the invention.

### EXAMPLES

### Example 1

Example 1 describes experiments that show the preparation of lipid nanoparticles in presence of a lyophilisation aid. In these experiments it has been verified that exemplary lyophilisation aid (sucrose) can be added directly to the second liquid composition (citrate buffer) without inhibiting lipid nanoparticle formation.

Preparation of solutions: A solution of lipids in ethanol (first liquid composition) and a citric acid solution with or without sucrose (second liquid composition) were prepared. The composition of the first liquid composition is listed in table 1. The lipids are dissolved in ethanol (≥95% analytical grade) at a total lipid concentration of 10 mg/ml according to mole fraction ratios of MC3/Chol/DSPC/DMG-PEG 2000—50/38.5/10/1.5 mol %.

TABLE 1

| | Molecular weight | Mole fraction | |
|---|---|---|---|
| Compound | [g/mol] | [Mol %] | Mass % |
| MC3 (D-Lin-MC3-DMA) | 642.09 | 50 | 54.7 |
| Chol (Cholesterol) | 386.65 | 38.5 | 25.4 |
| DSPC | 790.15 | 10 | 13.5 |
| DMG-PEG 2000 | 2509.2 | 1.5 | 6.4 |

The second liquid composition comprised 50 mM citrate buffer (pH 4) either with or without sucrose, i.e. with 40% (w/v) or 0% (w/v) sucrose, respectively.

PBS buffer pH 7.4 was prepared by first preparing a 10 liter stock of 10×PBS by dissolving 800 g NaCl, 20 g KCl, 144 g Na₂HPO₄*2H₂O, 24 g KH₂PO₄, and 8 l of distilled water. After complete mixing, the final solution was topped up to 10 l. As necessary, the pH was adjusted to 7.4 using hydrochloric acid or sodium hydroxide. The resulting PBS buffer had a final concentration of 137 mM NaCl, 10 mM phosphate, 2.7 mM KCl, and a pH of 7.4. Subsequently, to some of the buffer sucrose was added to achieve a sucrose concentration of 40% (w/v).

Production of lipid nanoparticles: Using a jet impingement reactor (MJR-type), the solution of lipids in ethanol and the citrate buffer were mixed. The nozzle size (diameter) of the reactor was 100 m at the first fluid inlet of the first stream and 200 m at the second fluid inlet of the second stream (i.e. 100/200). The total flow rate (TFR) of the streams was 50 ml/min. The volume flow ratio of the lipid solution (first stream, flow rate 1) to that of the aqueous buffer (second stream, flow rate 2), also referred to as the flow rate ratio (FRR) was 1:2. The reactor was connected to an apparatus providing the containers, tubing, pumps, valves, pressure gauges, thermometers and flow meters required to operate the reactor. The mixing resulted in a liquid composition (third liquid composition) comprising lipid nanoparticles, which was collected and characterised. An overview of the different batches of the samples is given in table 2.

TABLE 2

| | Nozzle | | | Flow | Flow | Sucrose |
|---|---|---|---|---|---|---|
| Batch | sizes | | TFR | rate 1 | rate 2 | in stream 2 |
| ID | [μm] | FRR | [ml/min] | [ml/min] | [ml/min] | [w/v] |
| 1.1 | 100/200 | 1:2 | 50 | 16.7 | 33.3 | 0% |
| 1.2 | 100/200 | 1:2 | 50 | 16.7 | 33.3 | 0% |
| 1.3 | 100/200 | 1:2 | 50 | 16.7 | 33.3 | 0% |
| 2.1 | 100/200 | 1:2 | 50 | 16.7 | 33.3 | 40% |
| 2.2 | 100/200 | 1:2 | 50 | 16.7 | 33.3 | 40% |
| 2.3 | 100/200 | 1:2 | 50 | 16.7 | 33.3 | 40% |

Dilution of the samples: The samples were diluted down to 10% ethanol with 50 mM citrate buffer containing the respective sucrose concentrations immediately after production, since high amounts of ethanol can destabilize the LNPs. A sufficient fraction of the sample before dilution was kept aside for particle characterization before the dilution. Samples were stored on ice until filled in dialysis cassettes.

Dialysis: In preparation of the particle characterisation, a dialysis step was performed that eliminates the ethanol. The samples were filled into separate dialysis cassettes (sample volume 10 ml) and dialyzed against PBS containing the same sucrose concentration as was used for the second stream (i.e. 0% and 40% sucrose) with moderate magnetic stirring. The buffer was exchanged after ca. 2 h, after 4 h, and then the samples where dialyzed overnight Particle characterization: Particle formation was verified by analysing the average particle size, also referred to as the “z-average diameter”. The z-average diameter may be measured by Dynamic Light Scattering (DLS), a technique commonly known to be used to determine the size distribution profile of small particles in suspension. For the measurements, a Zetasizer Nano ZS from Malvern Panalytical Ltd was used. For the determination of the average particle size, the temperature was kept constant at 25° C. during the measurement. The z-average diameter, together with the polydispersity index (PDI), was calculated from the cumulants analysis of the DLS measured intensity autocorrelation function as defined in ISO22412:2008. PDI is a dimensionless estimate of the width of the particle size distribution, scaled from 0 to 1.

Particle formation prepared with addition of sucrose and without sucrose were compared before and after dialysis. Results are given in table 3.

TABLE 3

| | Before dialysis | | | After dialysis | | |
|---|---|---|---|---|---|---|
| Batch | | z-average | | | z-average | |
| ID | Observation | [nm] | PDI | Observation | [nm] | PDI |
| 1.1 | | 222.5 | 0.043 | Small peak | 76.83 | 0.13 |
| 1.2 | | 235.7 | 0.11 | at 5 μm | 80.29 | 0.155 |
| 1.3 | | 247.9 | 0.114 | | 81.49 | 0.112 |
| 2.1 | | 505.8 | 0.611 | Small peak | 224.3 | 0.146 |
| | | | | at 5 μm | | |
| 2.2 | | 374.5 | 0.428 | Small peak | 246.5 | 0.161 |
| 2.3 | | 1.499e4 | 0.453 | at 5 μm | 275.3 | 0.156 |

Result: Example 1 showed that the formation of lipid nanoparticles is possible even in the presence of a high concentration of a lyophilisation aid like sucrose. It could also be shown that a subsequent dialysis step is not necessary for the formation of particles.

### Example 2

In a procedure similar to example 1 but using a lower sucrose concentration, lipid nanoparticles were prepared. Moreover, the dialysis step to remove the ethanol was performed with either PBS with 10% sucrose or without sucrose.

Preparation of solutions: The same solutions were used as in example 1 with the following exception: The second liquid composition comprised 50 mM citrate buffer (pH 4) and 10% (w/v) sucrose. The dialysis buffer PBS contained sucrose at concentrations of 0% and 10% (w/v).

Preparation of lipid nanoparticles: The lipid nanoparticles were prepared as described in example 1. An overview of the different batches of the samples is given in table 3.

TABLE 3

| | | | | | | Sucrose | Sucrose |
|---|---|---|---|---|---|---|---|
| | Nozzle | | TFR | Flow | Flow | in | in dialysis |
| Batch | sizes | | [ml/ | rate 1 | rate 2 | stream 2 | buffer |
| ID | [μm] | FRR | min] | [ml/min] | [ml/min] | [w/v] | [w/v] |
| 1.1 | 100/200 | 1:2 | 50 | 16.7 | 33.3 | 10% | Not dialized |
| 1.1a | 100/200 | 1:2 | 50 | 16.7 | 33.3 | 10% | 0% |
| 1.1b | 100/200 | 1:2 | 50 | 16.7 | 33.3 | 10% | 10% |
| 1.2 | 100/200 | 1:2 | 50 | 16.7 | 33.3 | 10% | Not dialized |
| 1.2a | 100/200 | 1:2 | 50 | 16.7 | 33.3 | 10% | 0% |
| 1.2b | 100/200 | 1:2 | 50 | 16.7 | 33.3 | 10% | 10% |

Dilution of the samples: The samples were diluted as described for example 1.

Dialysis: For particle characterization, the samples were filled into dialysis cassettes. 5 ml of each sample were dialyzed against PBS with no sucrose added and another 5 ml were dialyzed against PBS with 10% sucrose; both with moderate magnetic stirring. The buffer was exchanged after ca. 2 h and after 4 h, and then the samples where dialyzed overnight.

Particle characterization: The particle characterization was carried out as described above for example 1. Results are given in table 4.

TABLE 4

| | Before dialysis | | | After dialysis | | |
|---|---|---|---|---|---|---|
| Batch | | z-average | | | z-average | |
| ID | Observation | [nm] | PDI | Observation | [nm] | PDI |

| | | | | |
|---|---|---|---|---|
| 1 | Small peak | 144.4 | 0.197 | |
| 1.a | at 5 μm | | | 109.4 |
| 1.b | | | | 110.1 |
| 2 | | 132.2 | 0.122 | |
| 2.a | | | | 110.0 |
| 2.b | | | | 109.4 |

Result: Lipid nanoparticles had an average size of about 110 nm and a PDI of <0.2 after dialysis. It was shown that lipid nanoparticles could be prepared in the presence of 10% [w/v] as lyophilisation aid, and that the presence of sucrose in a subsequent dialysis step has little impact on the size of the lipid nanoparticles. It is believed that these findings support the gist of the present invention in that they show that the preparation of lipid nanoparticles and their subsequent conversion into a lyophilisate may actually be integrated efficiently within a single process.

### Example 3

Lipid nanoparticles encapsulating poly(A) (polyadenylic acid) was prepared in a procedure similar to the procedure described in Example 1, and using a jet impingement reactor, with the second liquid composition comprising poly(A), 50 mM citrate buffer (pH 4), and 10% (w/v) of a lyophilisation aid (a combination of sucrose (60%) and dextran (40%)). The resulting lipid nanoparticle composition (with 25 vol. % ethanol) was directly filled into glass vials without further addition or removal of constituents. Three sample vials (0.5 mL fill volume) were prepared. The filled vials were placed into a freeze dryer and freeze-dried for an initial period of at least 24 h at −55° C., and ca. 25 bar to obtain a lyophilized composition. The cake aspect of the freeze-dried samples was visually evaluated immediately after freeze-drying and scored a value of 0, 1 or 2, with 0 indicating no cake present or cake located at the top of the vial, 1 indicating cake formation at the bottom or side of vial, but with some visible defects, and 2 indicating an acceptable cake structure. The three tested samples were each scored a value of 2, and were visually found to be comparable to lyophilized control samples comprising no lipid nanoparticles but only citrate buffer, ethanol and lyophilization aid, demonstrating that cake formation of the lipid nanoparticle composition is feasible even at the relatively higher ethanol content of the composition.

### Example 4

Example 4 is a prophetic approach to the preparation of lipid nanoparticles using the method of the invention that includes a freeze-drying step. It is considered to perform the same steps as in example 2 (in presence of 10% sucrose). The steps of dilution of the samples and dialysis of the above example 1 will be replaced by a direct filling of the third liquid composition comprising the placebo lipid nanoparticles into 2 ml injection vials (2R vial) made of pharmaceutical grade tubular glass, with a crimp neck (e.g. from APG Europe, Dortmund Germany) and a subsequent freeze drying step. The freeze-drying step is performed in a laboratory freeze-dryer, like Alpha 1-4 LSCplus (Martin Christ Gefriertrocknungsanlagen GmbH, Osterode am Harz, Germany). For particle characterisation, the dried sample will be brought into solution with help of a buffer at an pH about 9 in order to neutralize the acidic pH of the sample that was present before freeze drying due to the use of citric acid. It is expected that lipid nanoparticles with a z-average particle size of 50 nm to 500 nm and a PDI below 0.3 or even below 0.2 are obtained.

### DESCRIPTION OF THE DRAWINGS

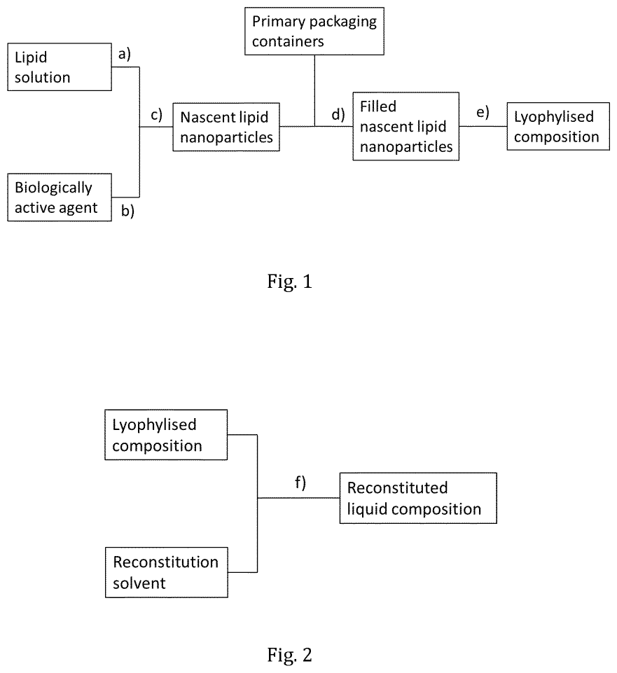

FIG. 1 shows a schematic overview according to one embodiment of the method for preparing a lipid nanoparticle composition. In step a) an organic solution of one or more lipids (lipid solution) is provided. In step b) a biologically active agent in an aqueous solution is provided. These two solutions are mixed under formation of a composition comprising the nascent lipid nanoparticles in step c), which is then filled into primary packaging containers in step d). Subsequently in step e), the containers filled with the mixture comprising the nascent lipid nanoparticles are subjected to a freeze drying process such as to obtain a lyophilised composition.

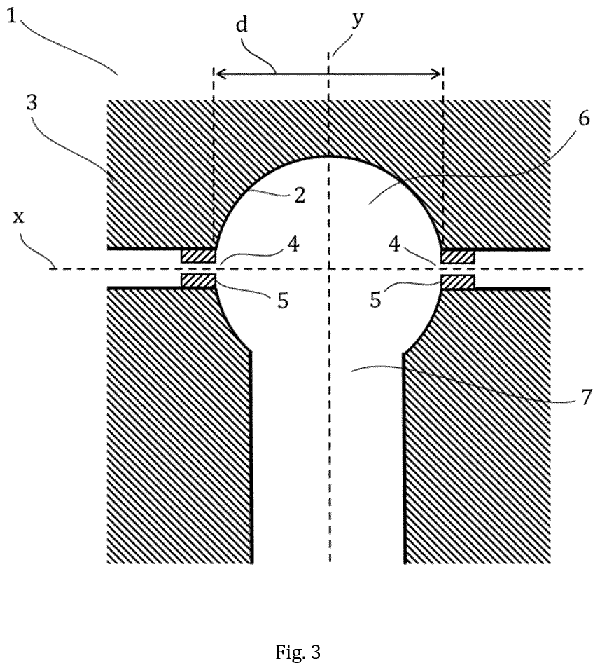

FIG. 2 shows schematically the reconstitution step according to one embodiment of the method for preparing a lipid nanoparticle composition. The lyophilised composition is brought together with a liquid reconstitution solvent (step f) such as to obtain the reconstituted liquid composition comprising the lipid nanoparticles and the biologically active agent associated with and/or encapsulated within the lipid nanoparticles.

FIG. 3, which is not to scale, depicts a jet impingement reactor used in one of the embodiments of the method of the invention. The jet impingement reactor (1) comprises a mixing chamber (6) having a substantially spherical shape. The mixing chamber (6) is defined by the interior surface (2) of the chamber wall (3), except for the two fluid inlets (4) and the fluid outlet (7). The fluid inlets (4) are arranged at opposite positions on a first central axis (x) of the reaction chamber (6) and point at one another. Each of the fluid inlets (4) comprises a nozzle (5), which is a plain orifice nozzle in this embodiment. The fluid outlet (7) is positioned on a second central axis (y) which is perpendicular to the first central axis (x). The distance (d) between the two nozzles (4) is substantially the same as the diameter of the spherical reaction chamber (6).

1. A method of preparing a composition comprising lipid nanoparticles and a biologically active agent selected from oligo- and polynucleotides, wherein the biologically active agent is associated with and/or encapsulated within the lipid nanoparticles, comprising the steps of:
(a) providing a first stream of a first liquid composition, wherein the first liquid composition comprises an organic solution of one or more lipids;
(b) providing a second stream of a second liquid composition, wherein the second liquid composition comprises an aqueous solution of the biologically active agent;
(c) mixing the first stream and the second stream such as to form a third stream of a third liquid composition, wherein the third liquid composition comprises nascent lipid nanoparticles comprising the one or more lipids;
(d) directly filling the third liquid composition into a primary packaging container; and
(e) subjecting the primary packaging container to freeze drying such as to obtain a lyophilised composition;
wherein between step (c) and step (d) no step of removal or addition of a constituent from or to the third liquid composition is conducted; and
wherein steps (a) to (e) are conducted under aseptic conditions.

2. The method of claim 1, further comprising a step of
(f) reconstituting the lyophilised composition obtained in step (e) by combining it with a liquid reconstitution solvent such as to obtain a reconstituted liquid composition comprising the lipid nanoparticles and the biologically active agent associated with and/or encapsulated within the lipid nanoparticles, wherein the liquid reconstitution solvent is preferably a buffered aqueous liquid.

3. The method of any one of the preceding claims, wherein the biologically active agent is an RNA molecule, wherein the RNA molecule is preferably selected from siRNA and mRNA, and wherein the mRNA is optionally a modified RNA.

4. The method of any one of the preceding claims, wherein the one or more lipids comprise:
a cationic or cationisable lipid, wherein the cationic or cationisable lipid is preferably selected from the group of N1-[2-((1S)-1-[(3-aminopropyl)amino]-4-[di(3-amino-propyl)amino]butylcarboxamido)ethyl]-3,4-di[oleyloxy]-benzamide (MVL5), N4-cholesteryl-spermine HCl salt (GL67), N-(4-carboxybenzyl)-N,N-dimethyl-2,3-bis(oleoyloxy)propan-1-aminium (DOBAQ), 1,2-distearoyl-3-dimethylammonium-propane (18:0 DAP), 1,2-dipalmitoyl-3-dimethylammonium-propane (16:0 DAP), 1,2-dimyristoyl-3-dimethylammonium-propane (14:0 DAP), 1,2-dioleoyl-3-dimethylammonium-propane (DODAP) (18:1 DAP), 1,2-dioleyloxy-3-dimethylaminopropane (DODMA), 1,2-di-O-octadecenyl-3-trimethylammonium propane (chloride salt) (DOTMA), 1,2-dioleoyloxy-3-trimethylammonium-propane (chloride salt) (18:1 TAP or DOTAP), 1,2-stearoyl-3-trimethylammonium-propane (chloride salt) (18:0 TAP), 1,2-dipalmitoyl-3-trimethylammonium-propane (chloride salt) (16:0 TAP), 1,2-dimyristoyl-3-trimethylammonium-propane (chloride salt) (14:0 TAP), dimethyldioctadecylammonium (bromide salt) (18:0 DDAB), 1,2-dimyristoleoyl-sn-glycero-3-ethylphosphocholine (Tf salt) (14:1 EPC Tf Salt), 1-palmitoyl-2-oleoyl-sn-glycero-3-ethylphosphocholine (chloride salt) (16:0-18:1 EPC Cl salt), 1,2-dioleoyl-sn-glycero-3-ethylphosphocholine (chloride salt) (18:1 EPC Cl salt), 1,2-distearoyl-sn-glycero-3-ethylphosphocholine (chloride salt) (18:0 EPC Cl salt), 1,2-dipalmitoyl-sn-glycero-3-ethylphosphocholine (chloride salt) (16:0 EPC Cl salt), 1,2-dimyristoyl-sn-glycero-3-ethylphosphocholine (chloride salt) (14:0 EPC Cl salt), 1,2-dilauroyl-sn-glycero-3-ethylphosphocholine (chloride salt) (12:0 EPC Cl Salt), O,O′-ditetradecanoyl-N-(α-trimethylammonioacetyl)diethanolamine chloride (DC-6-14), 3ß-[N-(N′,N′-dimethylaminoethane)-carbamoyl]cholesterol hydrochloride (DC-cholesterol-HCl), 4-(dimethylamino)-butanoic acid, (10Z,13Z)-1-(9Z,12Z)-9,12-octadecadien-1-yl-10,13-nonadecadien-1-yl ester (D-Lin-MC3-DMA), ([(4-hydroxybutyl)azanediyl]di(hexane-6,1-diyl) bis(2-hexyldecanoate)) (ALC-0315), 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), cetyl-trimethyl ammonium bromide (CTAB), penta-amine N15-cholesteryloxycarbonyl-3,712-triazapentadecane-115-diamine (CTAP), bis-guanidinium-spermidine-Chol (BGSC), bis-guanidinium-tren-Chol (BGTC), N,N-distearyl-N-methyl-N-2[N′-(N2-guanidino-L-lysinyl)] aminoethyl ammonium chloride (DSGLA), O-(2R-1,2-di-O-(1Z,9Z-octadecadienyl)-glycerol)-3-N-(bis-2-aminoethyl)carbamate) (BTCA), 1,2-dilinoleyloxy-3-dimethylaminopropane (DLinDMA), N-[6-amino-1-oxo-1-(N-tetradecylamino)hexan-(2S)-2-yl]-N′-{2-[N,N-bis(2-aminoethyl)-amino]ethyl}-2,2-ditetradecylpropandiamide (DiTT4), cholesteryloxypropan-1-amine (COPA), and cholesteryl-2-aminoethylcarbamate (CAEC);
a PEGylated lipid, wherein the PEGylated lipid is preferably selected from the group of 1,2-dimyristoyl-rac-glycero-3-methoxypolyethylene glycol-2000 (DMG-PEG 2000), distearoyl-rac-glycerol-PEG2K (DSG-PEG 2000), 2 [(polyethylene glycol)-2000]-N,N-ditetradecylacetamid (ALC-0159), N-(carbonyl-methoxypolyethylenglycol 2000)-1,2-dimyristoyl-sn-glycero-3-phosphoethanolamine sodium salt (MPEG-2000-DMPE Na), N-(carbonyl-methoxypolyethylenglycol 750)-1,2-distearoyl-sn-glycero-3-phosphoethanolamine sodium salt (MPEG-750-DSPE), 1,2-distearoyl-sn-glycero-3-phosphoethanolamine-N-[amino(polyethylene glycol)-2000] (ammonium salt) (DSPE-PEG(2000)), 1,2-dipalmitoyl-sn-glycero-3-phosphoethanolamine-N-[methoxy(polyethylene glycol)-2000] (ammonium salt) (16:0 PEG2000 PE), 1,2-dimyristoyl-sn-glycero-3-phosphoethanolamine-N-[methoxy(polyethylene glycol)-2000] (ammonium salt) (14:0 PEG2000 PE), 1,2-dioleoyl-sn-glycero-3-phosphoethanolamine-N-[methoxy(polyethylene glycol)-2000] (ammonium salt) (18:1 PEG2000 PE), 1,2-distearoyl-sn-glycero-3-phosphoethanolamine-N-[methoxy(polyethylene glycol)-2000] (ammonium salt) (18:0 PEG2000 PE), 1,2-distearoyl-sn-glycero-3-phosphoethanolamine-N-[methoxy(polyethylene glycol)-5000](ammonium salt) (18:0 PEG5000 PE), N-(carbonyl-methoxypolyethylenglycol 2000)-1,2-dipalmitoyl-sn-glycero-3-phosphoethanolamine sodium salt (MPEG-2000-DPPE Na);
a non-PEGylated zwitterionic lipid, wherein the non-PEGylated zwitterionic lipid is preferably selected from the group of 1,2-dimyristoyl-sn-glycero-3-phosphocholine (DMPC), 1,2-dioleoyl-sn-glycero-3-phosphoethanolamine (18:1 (A9-cis) PE or DOPE), 1,2-Dipalmitoyl-sn-glycero-3-phosphate (sodium salt) (DPPA, Na), 1-palmitoyl-2-oleoyl-sn-glycero-3-phosphocholine (POPC), 1,2-dipalmitoyl-sn-glycero-3-phosphocholine (16:0 PC or DPPC), 1,2-distearoyl-sn-glycero-3-phosphocholine (DSPC), 1,2-dimyristoyl-sn-glycero-3-phosphatidylethanolamine (DMPE), 1-palmitoyl-2-oleoyl-sn-glycero-3-phosphatidylethanolamine (1,2-POPE), cholesterol 3-sulfate (sodium salt) (SCS), 1,2-dioleoyl-sn-glycerol-3-phosphatidylcholine (DOPC), and trans-2-aminoacyclohexanol (TACH); and/or
cholesterol.

5. The method of any one of the preceding claims, wherein the first liquid composition comprises a water-miscible solvent, wherein the water-miscible solvent is preferably selected from ethanol, methanol, acetone, acetonitrile, acetic acid, formic acid, trifluoroacetic acid, acetaldehyde, n-butanol, ethylamine, and any combinations thereof.

6. The method of any one of the preceding claims, wherein the total concentration of lipids in the first liquid composition is from about 20 to about 100 mg/mL, and optionally from about 50 to about 100 mg/mL.

7. The method of claim 5 or 6, wherein the first liquid composition essentially consists of a solution of the one or more lipids and optionally one or more further lipophilic excipients in the water-miscible solvent.

8. The method of claim 7, wherein the first liquid composition essentially consists of a solution of a cationic or cationisable lipid, a PEGylated lipid, a non-PEGylated zwitterionic lipid, cholesterol, and optionally one or more further lipophilic excipients in ethanol.

9. The method of any one of the preceding claims, wherein the second liquid composition comprises an osmotic agent, and wherein the osmotic agent is optionally selected from sodium chloride, potassium chloride, sorbitol, mannitol, and sucrose and/or wherein the second liquid composition comprises a lyophilisation aid, and wherein the lyophilisation aid is optionally selected from sucrose, trehalose, mannitol, sorbitol, glucose, fructose, proline, glycine betaine, polyethylene glycol, starch and dextran.

10. The method of any one of the preceding claims, wherein the first stream is provided in step (a) and/or the second stream is provided in step (b) at a pressure in the range of 10 kPa (0.1 bar) to 12,000 kPa (120 bar) and/or wherein the first stream is provided in step (a) and/or the second stream is provided in step (b) at a flow rate in the range of 1 to 1,000 ml/min, and wherein the flow rate of the second stream is preferably higher than the flow rate of the first stream.

11. The method of claim 10, wherein the flow rate of the second stream in step (b) is from about 2 times to about 4 times higher than the flow rate of first stream in step (a).

12. The method of any one of the preceding claims, wherein the primary packaging container is empty when provided for filling according to step (d).

13. The method of any one of claims 1 to 12, wherein the primary packaging container contains an amount of a fourth liquid composition when provided for filling according to step (d), and wherein step (d) results in the mixing of the third and the fourth liquid composition such as to form a fifth liquid composition.

14. The method of claim 13, wherein the fourth liquid composition is preferably an aqueous solution comprising
a pH-regulating agent;
an osmotic agent; and/or
a lyophilisation aid.

15. The method of claim 13 or 14, wherein the amount of the fourth liquid composition in the primary packaging container when provided for filling according to step (d) is from about 50 to about 150 wt % relative to the amount of the third liquid composition filled into the primary packaging container in step (d).

16. The method of any one of claims 13 to 15, wherein the fifth liquid composition is an aqueous composition comprising lipid nanoparticles, further comprising from about 5 to about 20 vol. % organic solvent, wherein the organic solvent is preferably ethanol.

17. The method of any one of claims 13 to 16, wherein the fifth liquid composition is an aqueous composition comprising from about 5 to about 35 wt %, and preferably from about 10 to about 25 wt % of a lyophilisation aid.

18. The method of any one of the preceding claims, wherein the mixing of the first stream and the second stream in step (c) is performed using a T-piece, a Y-piece, a static mixer, or a microfluidic mixer.

19. The method of claim 18, wherein the static mixer comprises a mixing chamber (6) into which each of the first and the second stream are injected through a nozzle (5) such that said streams collide with one another frontally in the mixing chamber (6).

20. The method of claim 19, wherein the mixing chamber is part of a jet impingement reactor (1), defined by an interior surface (2) of a mixing chamber wall (3), the mixing chamber (6) having a substantially spheroidal overall shape and wherein the mixing chamber (6) preferably comprises
(a) a first and a second fluid inlet (4), wherein the first and the second fluid inlet (4) are arranged at opposite positions on a first central axis (x) of the reaction chamber (6) such as to point at one another, and wherein each of the first and the second fluid inlet (4) comprises a nozzle (5); and
(b) a fluid outlet (7) arranged at a third position, said third position being located on a second central axis (y) of said chamber (6), the second central axis (y) being perpendicular to the first central axis (x); and
wherein the distance (d) between the nozzle (5) of the first fluid inlet (4) and the nozzle (5) of the second fluid inlet (4) is the same or smaller than the diameter of the mixing chamber (6) along the first central axis (x).

21. A primary packaging container comprising the lyophilised composition as obtained in step (e) of the method according to any of the claims 1 to 20.

22. A reconstituted lyophilised composition as obtained in step (f) of the method according to any of the claims 2 to 20.

23. A kit comprising at least a first kit component and a second kit component, wherein the first kit component comprises a primary packaging container according to claim 21, and wherein the second kit component comprises an amount of the liquid reconstitution solvent according to claim 2.

24. Use of the primary packaging container according to claim 21 or of the reconstituted lyophilised composition of claim 22 or of the kit of claim 23 in the manufacture of a medicament, wherein the medicament is preferably a vaccine.
